(19) United States
(12) Patent Application Publication
(10) Pub. No.: US 2023/0397638 A1
(43) Pub. Date: Dec. 14, 2023

(54) CONTROL DEVICE, CONTROL METHOD, AND PROGRAM

(71) Applicant: SONY GROUP CORPORATION, TOKYO (JP)

(72) Inventors: KAE NAGANO, TOKYO (JP); NOBUHIKO MUKAI, TOKYO (JP)

(21) Appl. No.: 18/251,395

(22) PCT Filed: Sep. 17, 2021
(86) PCT No.: PCT/JP2021/034366

§ 371 Date: May 1, 2023

(30) Foreign Application Priority Data
Nov. 9, 2020 (JP) 2020-186820

(51) Int. Cl. A23L 5/30; B05B 12/12; B05B 17/06

(52) U.S. Cl. CPC A23L 5/32; B05B 12/12; B05B 17/06

(57) ABSTRACT

A control device according to an embodiment of the present disclosure includes a controller that controls each generation of a mist from a plurality of foods.

### TECHNICAL FIELD

The present disclosure relates to a control device, a control method, and a program.

### BACKGROUND ART

In recent years, a technique for misting a liquid has been used in various fields (for example, Patent Literature 1).

### CITATION LIST

### Patent Literature

Patent Literature 1: Japanese Unexamined Patent Application Publication No. H07-123077

### SUMMARY OF THE INVENTION

Such a liquid misting technology has been used in a pharmaceutical field (e.g., a nebulizer, etc.) or an air conditioning field (e.g., a humidifier, etc.), but its use in other fields has not been sufficiently studied. Therefore, it is desirable to consider the use of the liquid misting technology in more diverse fields.

It is desirable to provide a control device, a control method, and a program that make it possible to provide a user with a more entertaining eating and drinking experience.

A control device according to an embodiment of the present disclosure includes a controller that controls each generation of a mist from a plurality of foods.

A control method according to an embodiment of the present disclosure includes controlling, with an arithmetic processing unit, each generation of a mist from a plurality of foods.

A program according to an embodiment of the present disclosure causes a computer to function as a controller that controls each generation of a mist from a plurality of foods.

The control device, the control method, and the program according to an embodiment of the present disclosure control each generation of the mist from the plurality of foods. As a result, for example, it is possible to mix the mists generated from the plurality of foods in chronological order, or to generate the mists of the plurality of foods in conjunction with the production of music, image, or the like.

### BRIEF DESCRIPTION OF THE DRAWINGS

FIG. 1 is an explanatory diagram illustrating an overview of a control device according to a first embodiment of the present disclosure.

FIG. 2 is a see-through perspective diagram illustrating an example of a configuration of a mist generating device.

FIG. 3 is a schematic longitudinal sectional diagram illustrating the mist generating device illustrated in FIG. 2.

FIG. 4 is a block diagram illustrating a connection of the control device according to the embodiment and the mist generating device.

FIG. 5 is a flowchart illustrating an example of operation of the control device.

FIG. 6 is an explanatory diagram illustrating an example of an image presented to a user from the control device when the user makes an input.

FIG. 7 is a flowchart illustrating an example of a control in the mist generating device.

FIG. 8 is a flowchart illustrating an example of a control in the mist generating device.

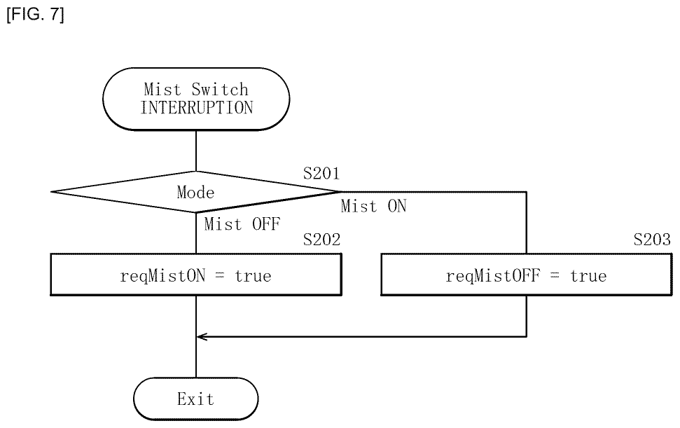

FIG. 7 is a see-through perspective diagram illustrating an example of a configuration of a mist generating device used in a second embodiment of the present disclosure.

FIG. 10 is a graph illustrating an example of changing an amount of mist generated from a plurality of foods on a time axis.

FIG. 11 is a flowchart illustrating an example of a control in the mist generating device.

FIG. 12A is a perspective diagram illustrating an example of a configuration of a mist generating device used in a third embodiment of the present disclosure.

FIG. 12B is a perspective diagram illustrating another example of the configuration of the mist generating device used in the embodiment.

FIG. 13 is a flowchart illustrating an example of a control in the mist generating device.

FIG. 14 is an explanatory diagram describing a modification example of the embodiment.

FIG. 15 is a perspective diagram illustrating an example of a configuration of a mist generating device used in a fourth embodiment of the present disclosure.

FIG. 16 is a schematic longitudinal sectional diagram illustrating the mist generating device illustrated in FIG. 15.

FIG. 17 is a schematic perspective diagram illustrating an appearance of an edible member enclosing food that generates the mist.

FIG. 18 is a longitudinal sectional diagram illustrating an internal structure of the edible member illustrated in FIG. 17.

FIG. 19 is a see-through perspective diagram illustrating a configuration of the mist generating device in which the edible member is placed in an oscillating tank.

FIG. 20 is an explanatory diagram illustrating a control by a control device according to a sixth embodiment of the present disclosure.

FIG. 21 is a block diagram illustrating an example of a hardware configuration of the control device according to each embodiment of the present disclosure.

### MODES FOR CARRYING OUT THE INVENTION

Hereinafter, embodiments of the present disclosure will be described in detail with reference to the drawings. The embodiments described below are specific examples of the present disclosure, and a technique according to the present disclosure is not limited to the following embodiments. In addition, the arrangement, dimensions, dimensional ratios, and the like of each component of the present disclosure are not limited to the embodiments illustrated in the drawings.

The description will be given in the following order.
1. First Embodiment
   1.1. Overview
   1.2. Configuration Example
   1.3. Operation Example
2. Second Embodiment
   2.1. Configuration Example
   2.2. Operation Example
3. Third Embodiment
   3.1. Configuration Example
   3.2. Operation Example
   3.3. Operation Example
4. Fourth Embodiment
5. Fifth Embodiment
6. Sixth Embodiment
7. Hardware Configuration Example

### 1. First Embodiment

### 1.1. Overview

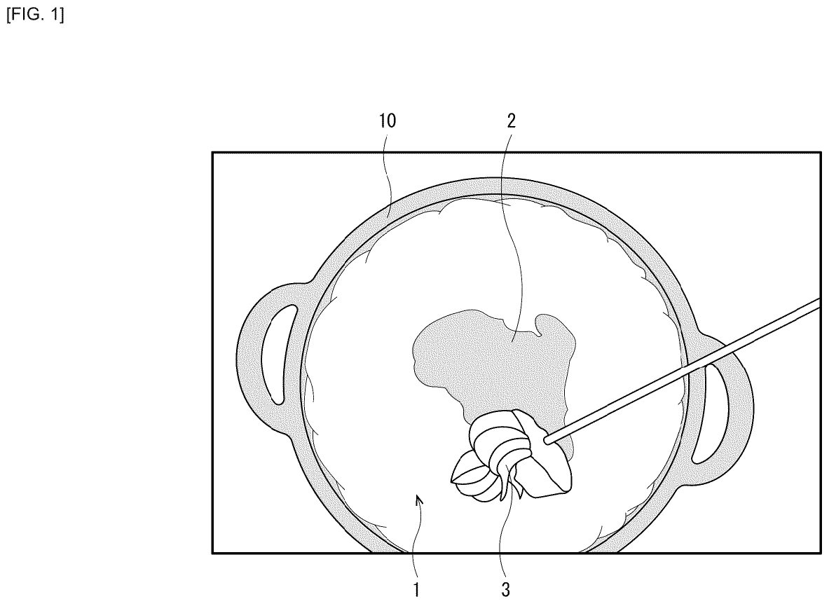

First, an overview of a control device according to a first embodiment of the present disclosure will be described with reference to FIG. 1. FIG. 1 is an explanatory diagram illustrating an overview of the control device according to the present embodiment.

The control device according to the present embodiment is a control device that controls generation of a mist 1 from a liquid food 2 in a mist generating device 10, as illustrated in FIG. 1. The food 2 in the mist generating device 10 is, for example, an edible liquid such as red wine.

The mist 1 is a collection of minute liquid droplets of several micrometers of the food 2. The mist 1 is generated by, for example, ultrasonically atomizing, heating, or spraying the food 2 in the mist generating device 10 under a control of the control device according to the present embodiment, and is gradually diffused from the inside of the mist generating device 10.

The control device according to the present embodiment can provide the user with a highly entertaining eating and drinking experience by generating the mist 1 of the edible food 2. For example, by generating the mist 1 of the edible food 2, the control device can, for example, make the user enjoy the aroma of the food 2 more strongly. In addition, the control device attaches the mist 1 to a secondary ingredient 3 such as sponge cake or whipped cream having adsorption properties, thereby allowing the user to enjoy the secondary ingredient 3 with the flavor of the food 2. Furthermore, the control device can allow the user to enjoy the secondary ingredient 3 flavored with the food 2 by dipping the secondary ingredient 3 into the food 2.

### 1.2. Configuration Example

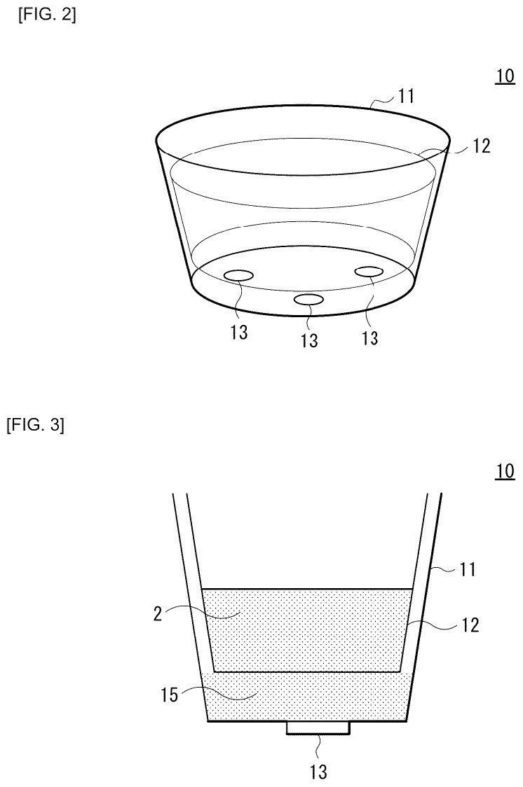

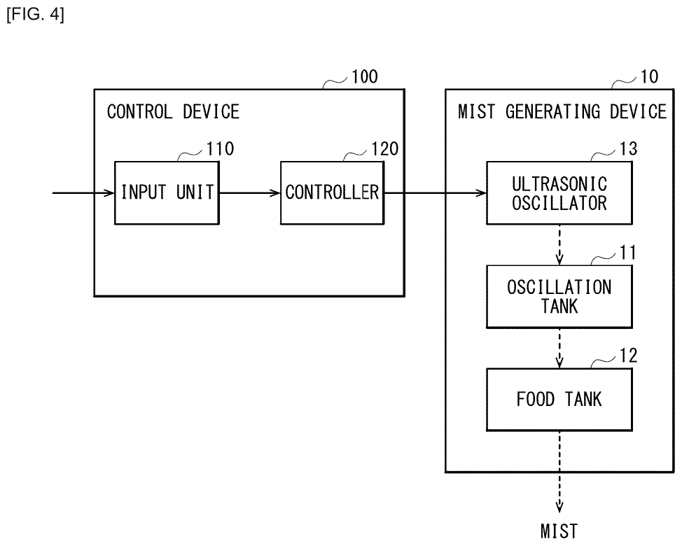

Next, a mist generating device controlled by the control device according to the present embodiment will be described with reference to FIGS. 2 to 4. FIG. 2 is a see-through perspective diagram illustrating an example of a configuration of a mist generating device 10. FIG. 3 is a schematic longitudinal sectional diagram illustrating the mist generating device 10 illustrated in FIG. 2. FIG. 4 is a block diagram illustrating a connection between the control device 100 according to the present embodiment and the mist generating device 10. However, a configuration of the mist generating device 10 controlled by the control device according to the present embodiment is not limited to the following configuration.

As illustrated in FIGS. 2 and 3, the mist generating device 10 includes an oscillation tank 11, a food tank 12, and an ultrasonic oscillator 13. The oscillation tank 11 contains a liquid 15 for propagating ultrasonic waves to the food tank 12, and the food tank 12 contains the food 2 to be misted.

The oscillation tank 11 has, for example, a plastic container-like structure (for example, a cup-like structure), and contains the liquid 15 inside the container-like structure. The liquid 15 is a medium for propagating the ultrasonic waves generated by the ultrasonic oscillator 13 provided at the bottom of the oscillation tank 11 to the food tank 12, and may be water, for example.

The food tank 12 has a plastic container-like structure (for example, a cup-like structure) with the area of a bottom part smaller than that of the oscillation tank 11, and is provided inside the oscillation tank 11 so that the bottom part is in contact with the liquid 15. Inside the container-like structure of the food tank 12, the liquid and edible food 2 is stored. The food 2 may be, for example, seasonings, alcoholic beverages, soup, or soup stock.

The liquid food 2 stored inside the container-like structure of the food tank 12 is misted by the ultrasonic waves that are generated by the ultrasonic oscillator 13 and propagated through the liquid 15 and the bottom part of the food tank 12. Specifically, in the liquid food 2, because the ultrasonic waves are irradiated from the inside of the liquid toward a liquid surface, a fountain-like liquid column is generated on the liquid surface due to a sound pressure, and small droplets of several micrometers are generated as the mist from a side surface of the liquid column. Such a phenomenon is also called ultrasonic atomization.

The ultrasonic oscillator 13 is provided at the bottom part of the oscillation tank 11 and includes an oscillator that generates the ultrasonic waves (for example, ultrasonic waves of several MHz to several tens of MHz) and a control board for the oscillator. The oscillator that generates the ultrasonic waves may be, for example, a piezoelectric ceramic element. The ultrasonic oscillator 13 may be provided on an inner side of the container-like structure of the oscillation tank 11 or may be provided an outer side of the container-like structure of the oscillation tank 11 (that is, on the back surface of the oscillation tank 11). The ultrasonic waves generated by the ultrasonic oscillator 13 propagate to the food 2 through the liquid 15 and the bottom part of the food tank 12.

The ultrasonic oscillator 13 may include a plurality of oscillators that generate ultrasonic waves with different or same frequencies. However, a size of droplets of the mist generated from the food 2 varies depending on the frequency of the irradiated ultrasonic waves, and an optimum frequency of the ultrasonic waves for misting varies depending on the viscosity of the food 2. Therefore, it is preferable that the ultrasonic oscillator 13 include a plurality of oscillators that generate the ultrasonic waves having different frequencies in order to make the frequency of the ultrasonic waves to be generated variable.

Moreover, the mist generating device 10 may further include a lid (not illustrated) that covers an opening surface of the food tank 12. The lid is a plate-like member that covers the opening surface of the food tank 12 and is detachably provided on the opening surface of the food tank 12.

The lid can prevent, for example, droplets of the liquid column rising from the liquid food 2 from scattering around the mist generating device 10 when the ultrasonic waves are generated from the ultrasonic oscillator 13. Further, the lid, for example, confines the generated mist of the food 2 inside the food tank 12, making it possible to achieve a performance in which, when the lid is removed from the mist generating device 10, the mist flows out all at once from the food tank 12.

As illustrated in FIG. 4, the control device 100 according to the present embodiment includes an input unit 110 and a controller 120.

The input unit 110 receives data used for controlling mist generation of the food 2 on the basis of an input from the user. Specifically, the input unit 110 may receive data such as a type of the food 2 to generate the mist, a type of the secondary ingredient 3 to which the mist of the food 2 is to be attached, or the time and timing for generating the mist. The input unit 110 may be an input device that receives the input from the user, such as a mouse, keyboard, touch panel, button, switch, or lever, or may be an input port that receives the input from the user inputted to an information processing device such as a mobile phone, a smartphone, a tablet terminal, or a personal computer.

The controller 120 controls the generation of the ultrasonic waves by the ultrasonic oscillator 13 of the mist generating device 10 on the basis of data received by the input unit 110, thereby controlling the generation of mist from the food 2. Specifically, the controller 120 may control the frequency of the ultrasonic waves to be generated by the ultrasonic oscillator 13, on the basis of at least one of the type of the food 2 for which the mist is to be generated or the type of the secondary ingredient 3 to which the mist of the food 2 is to be attached. In addition, the controller 120 may control the time and timing for generating the ultrasonic waves in the ultrasonic oscillator 13.

Furthermore, the controller 120 may control the mist generation from the food 2 on the basis of at least one or more of a state or an amount of the food 2. For example, the controller 120 may stop the generation of mist from the food 2 when the amount of the food 2 becomes equal to or less than a threshold. Further, in a case where the mist is to be generated from the food 2 that has been altered by the irradiation with the ultrasonic waves, the controller 120 may generate the mist from the food 2 after confirming that the food 2 has been sufficiently altered.

However, there is a time lag between the timing of the ultrasonic wave generation by the ultrasonic oscillator 13 and the timing of the mist generation from the food 2. Therefore, the controller 120 may control the timing of generating the ultrasonic waves by the ultrasonic oscillator 13 in consideration of the lag between the timing of generating the ultrasonic waves and the timing of generating the mist from the food 2. Furthermore, the lag between the timing of the ultrasonic wave generation and the timing of the mist generation from the food 2 may vary depending on the type of the food 2. Therefore, the controller 120 may control the timing of generating the ultrasonic waves in the ultrasonic oscillator 13 in accordance with the type of the food 2.

The ultrasonic waves generated by the ultrasonic oscillator 13 under the control of the controller 120 propagate to the food 2 via the oscillation tank 11, the liquid 15 contained in the oscillation tank 11, and the food tank 12. The ultrasonic waves propagated to the liquid food 2 generate a fountain-like liquid column on the liquid surface of the food 2 by the sound pressure, and the mist of the food 2 is generated from the side surface of the liquid column.

It should be noted that the connection between the control device 100 and the mist generating device 10 may be wired or wireless. For example, the control device 100 and the mist generating device 10 may be connected by wireless communication such as WiFi (registered trademark) or Bluetooth (registered trademark).

According to the above configuration, the control device 100 according to the present embodiment can control the generation of the mist from the food 2 by controlling the generation of the ultrasonic waves by the ultrasonic oscillator 13 of the mist generating device 10. The mist of the liquid food 2 generated by the mist generating device 10 can be used, for example, as entertainment related to eating and drinking to entertain the user with its aroma, or to entertain the user by attaching the mist to the secondary ingredient 3 to enjoy the taste.

### 1.3. Operation Example

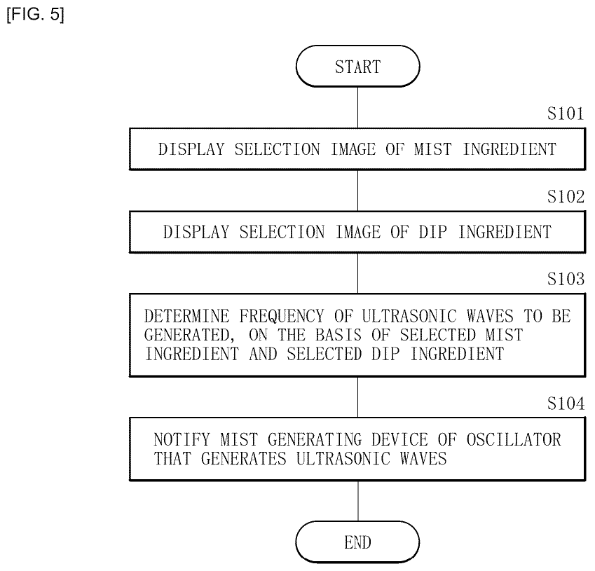

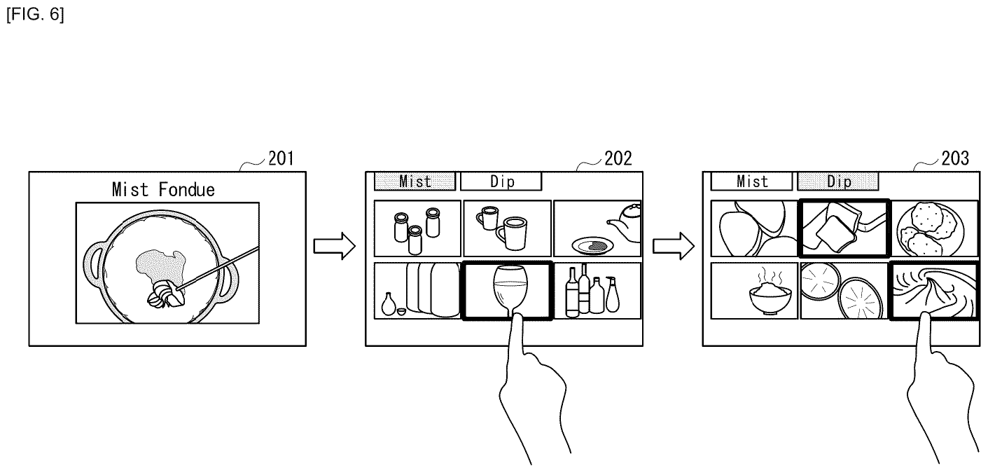

Subsequently, an operation of the control device 100 according to the present embodiment will be described with reference to FIGS. 5 and 6. FIG. 5 is a flowchart illustrating an example of the operation of the control device 100. FIG. 6 is an explanatory diagram illustrating an example of an image presented to the user from the control device 100 when the user makes an input. The control device 100 may, for example, use dedicated application software, images, etc., to allow the user to input the type of the food 2 to generate the mist and the type of the secondary ingredient 3 to which the mist of the food 2 is to be attached.

As illustrated in FIGS. 5 and 6, for example, the control device 100 displays a top page image 201 of the dedicated application software, and then displays a selection image 202 of a mist ingredient (S101). In the selection image 202, a plurality of image images of the food 2 (the mist ingredient) to be misted by the mist generating device 10 are displayed. For example, the selection image 202 displays the image images of milk, coffee, soy sauce, sake, wine, and vinegar.

After the food 2 to be misted is selected by the user from the selection image 202, the control device 100 displays a selection image 203 of a dip ingredient (S102). For the selection image 203, several types of image images of the secondary ingredient 3 (the dip ingredient) suitable for attaching or dipping of the food 2 selected in the selection image 202 are displayed. For example, for the selection image 203, image images of marshmallow, sponge cake, rice cracker, white rice, radish, and whipped cream are displayed.

Note that the secondary ingredient 3 displayed in the selection image 203 may be a secondary ingredient set in advance on a rule basis as the secondary ingredient 3 suitable for the attaching or dipping of the food 2 selected by the user, or may be a secondary ingredient derived from machine learning or the like.

After the user selects the secondary ingredient 3 from the selection image 203, the control device 100 determines the frequency of the ultrasonic waves to be generated by the ultrasonic oscillator 13, on the basis of the selected food 2 (the mist ingredient) and the secondary ingredient 3 (the dip ingredient) (S103). Specifically, the control device 100 determines the oscillator that generates the ultrasonic waves, on the basis of the types of the food 2 and the secondary ingredient 3 selected from among the plurality of oscillators that generate the ultrasonic waves of mutually different frequencies included in the ultrasonic oscillator 13. For example, the control device 100 determines, as the oscillator that generates the ultrasonic waves, the oscillator that generates the ultrasonic waves having a frequency that makes it possible to generate the mist from the selected food 2 and that makes it possible to generate the mist that includes droplets of a size suitable for attaching to the secondary ingredient 3.

Thereafter, the control device 100 notifies the mist generating device 10 of the frequency of the ultrasonic waves to be generated, etc. (S104). Specifically, the control device 100 notifies the ultrasonic oscillator 13 of the mist generating device 10 of the oscillator having been determined to generate the ultrasonic waves. As a result, the mist generating device 10 generates the ultrasonic waves with the oscillator notified from the control device 100, making it possible to generate the mist containing the droplets of an appropriate size from the food 2 stored in the food tank 12.

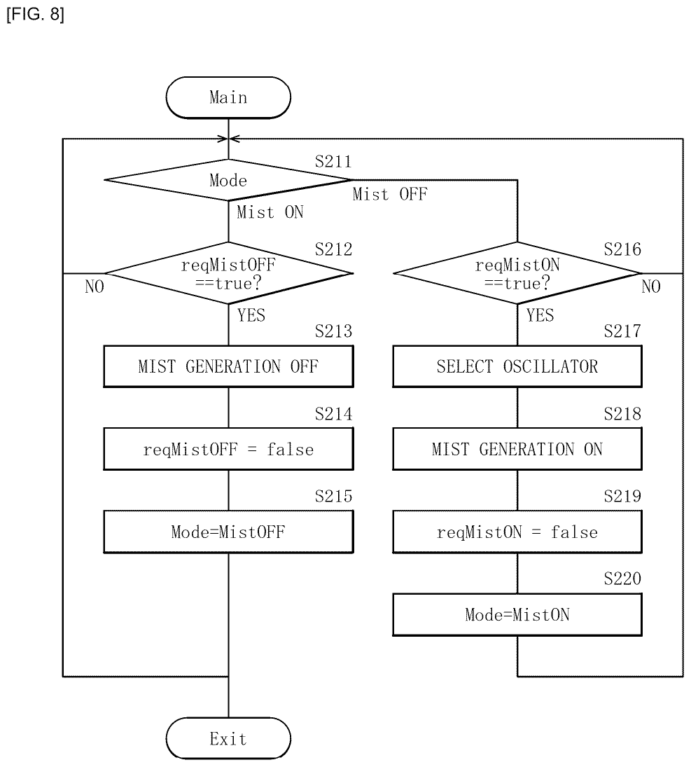

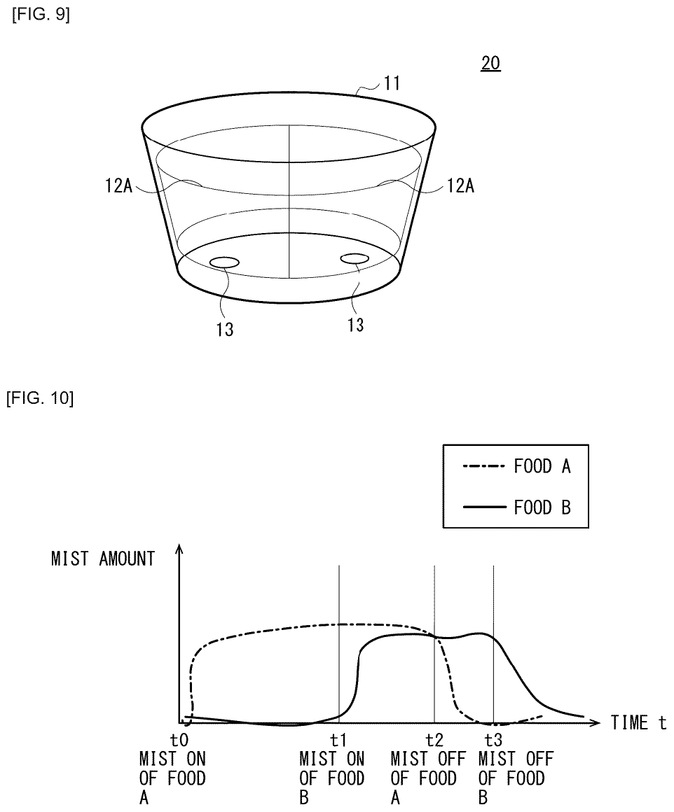

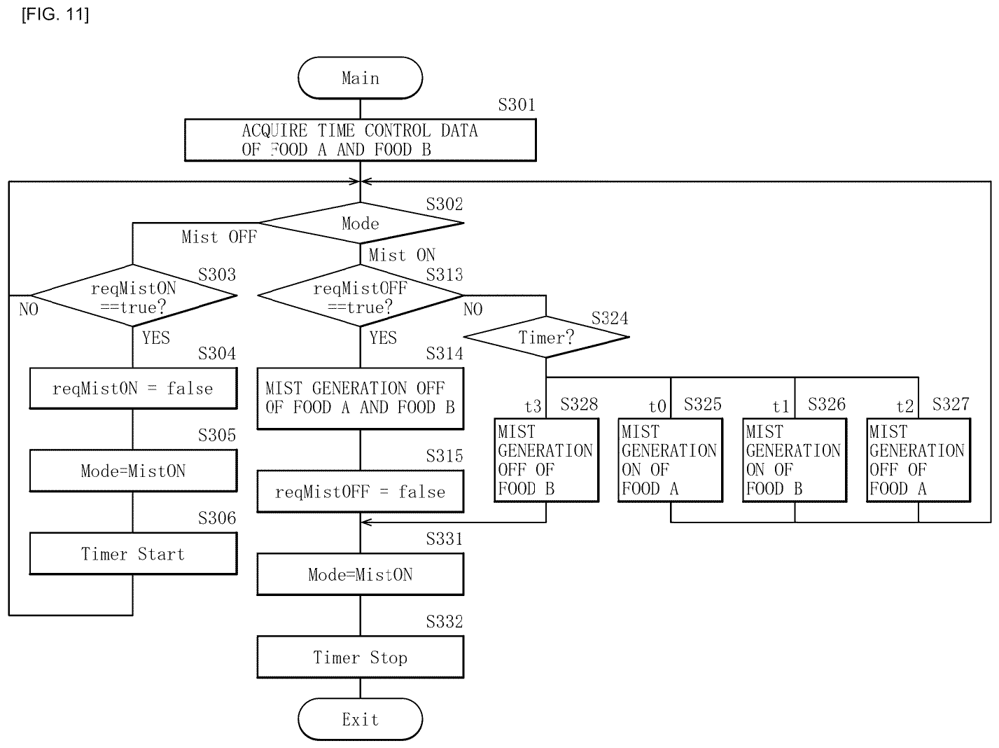

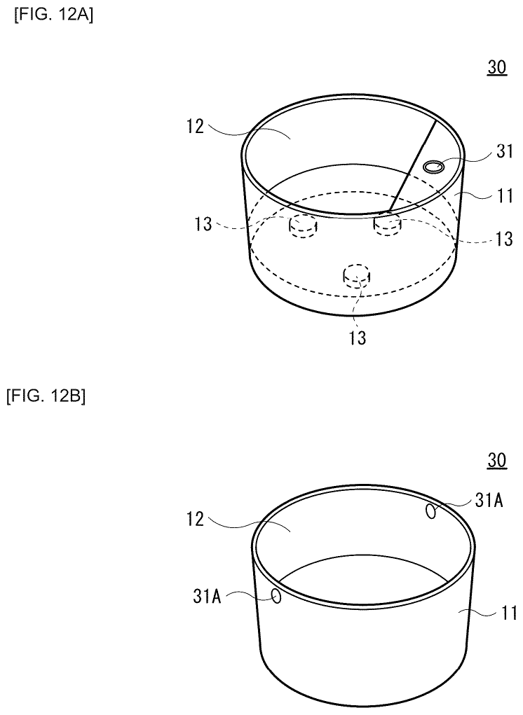

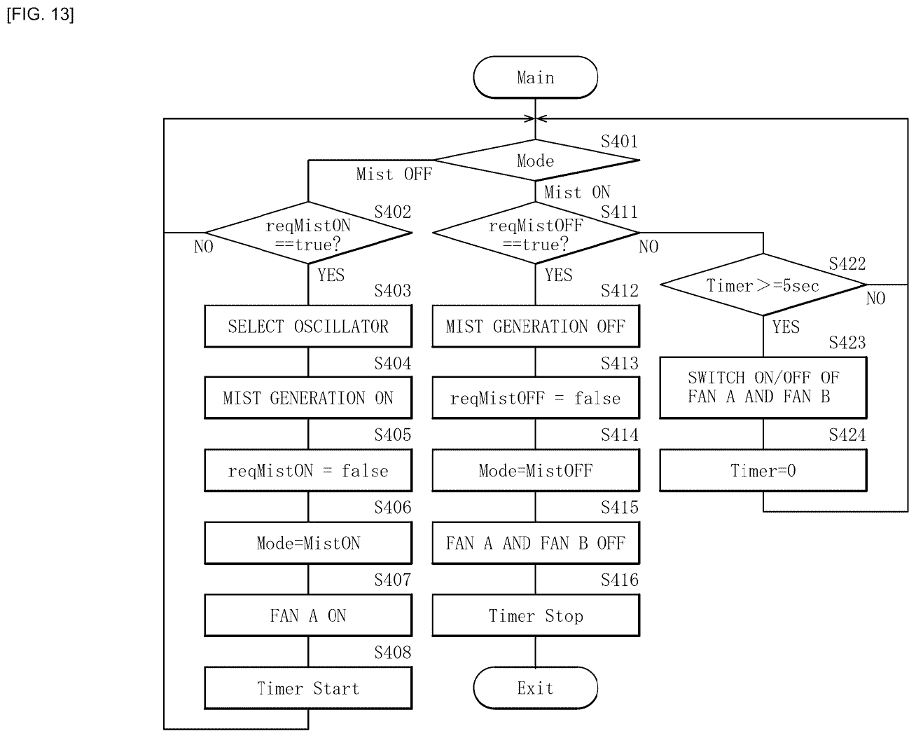

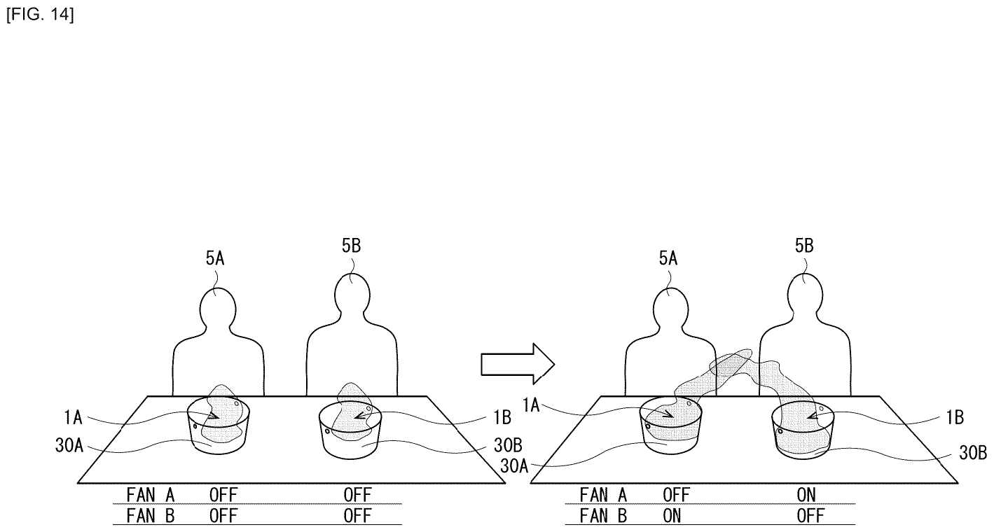

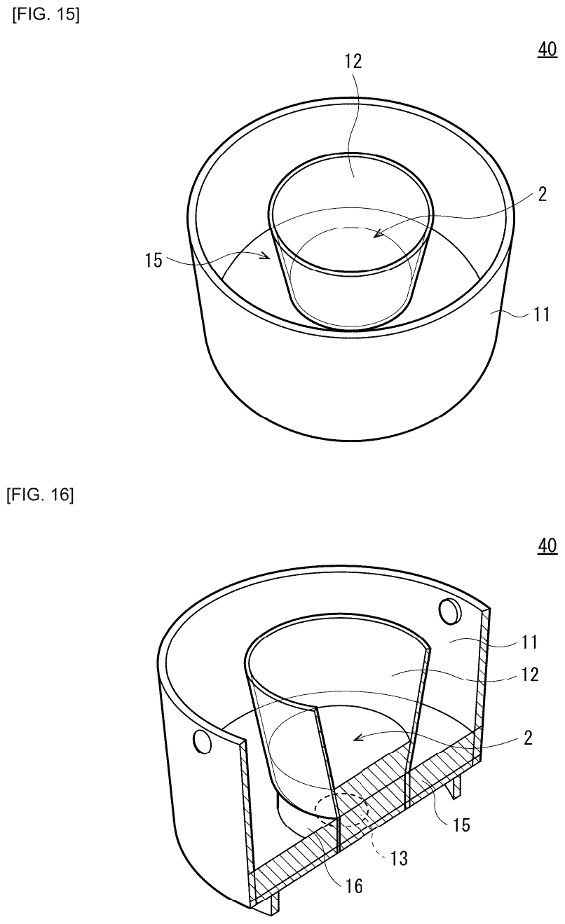

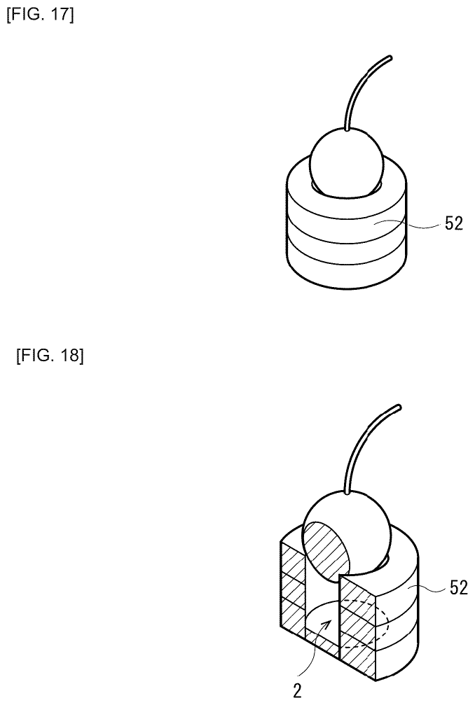

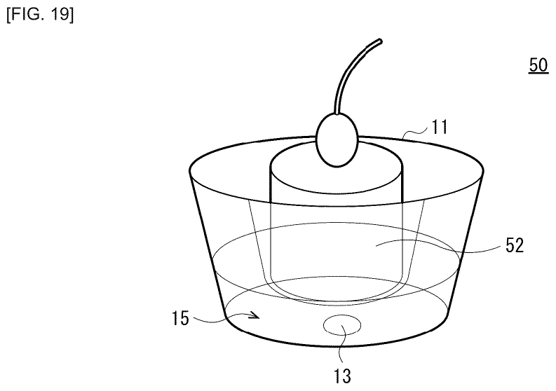

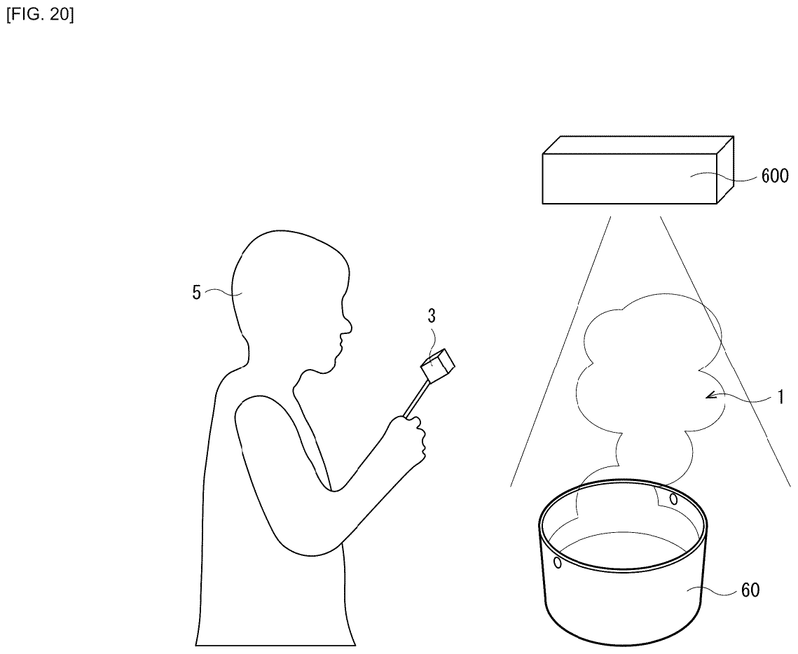

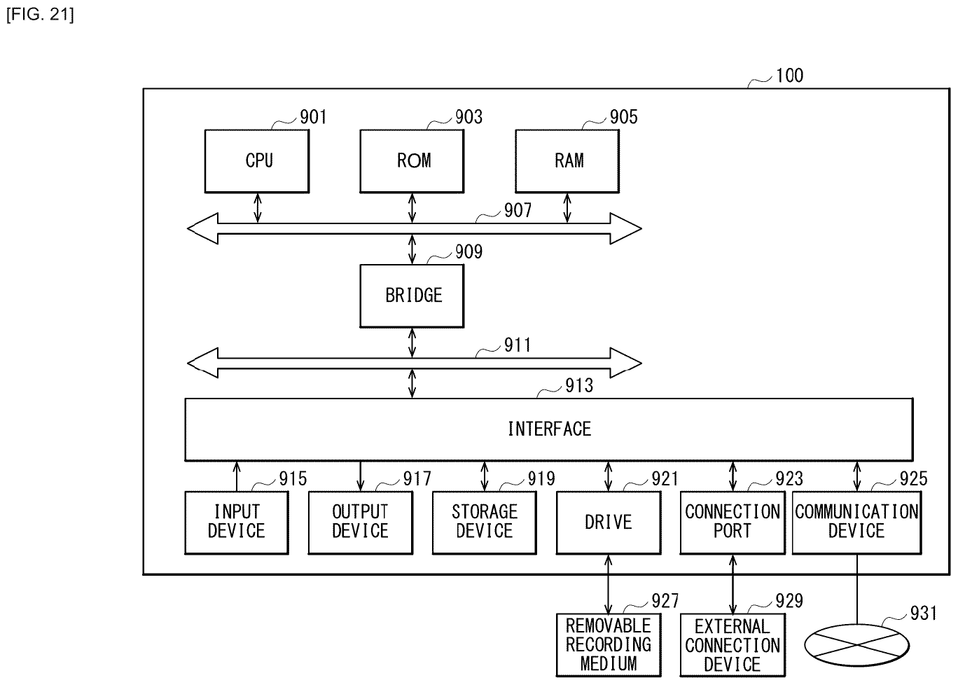

Furthermore, a control in the mist generating device 10 will be described with reference to FIGS. 7 and 8. FIGS. 7 and 8 are flowcharts illustrating an example of the control in the mist generating device 10.

As illustrated in FIG. 7, first, in the mist generating device 10, an interruption process to a system is performed by recognizing an input of a mist generation ON/OFF switch (Mist Switch) physically provided in the oscillation tank 11 or the like. Next, a mode of the system is checked (S201).

In a case where the mode of the system is in a mist ON state (Mist ON), the input of the mist generation ON/OFF switch is determined as an input to turn off the mist generation, and a mist OFF request flag is set (reqMistOFF=true). On the other hand, in a case where the mode of the system is in a mist generation OFF state (Mist OFF), the input of the mist generation ON/OFF switch is determined as an input to turn on the mist generation, and a mist ON request flag is set (reqMistON=true).

Subsequently, as illustrated in FIG. 8, the mode of the system is checked (S211).

In a case where the mode of the system is in the mist generation OFF state (Mist OFF), it is checked whether or not the mist ON request flag (reqMistON) is set (S216). If the mist ON request flag is set (S216/Yes), the ultrasonic oscillator of the ultrasonic waves notified from the control device 100 is selected (S217), and a mist generating circuit is controlled to be in an ON state (S218). Thereafter, the mist ON request flag is cleared (reqMistON=false, S219), and the mode of the system is set to the mist generation ON state (Mist ON).

On the other hand, in a case where the mode of the system is in the mist generation ON state (Mist ON), it is checked whether or not the mist OFF request flag (reqMistOFF) is set (S212). If the mist OFF request flag is set (S212/Yes), the mist generating circuit is controlled to be off (S213). Thereafter, the mist OFF request flag is cleared (reqMistOFF=false, S214), and the mode of the system is set to the mist generation OFF state (Mist OFF).

It should be noted that the control to turn off the mist generating circuit may be performed in a case where the liquid amount of the food 2 to be misted is equal to or less than a threshold value. According to this, the control device 100 can automatically stop the generation of the mist when the amount of liquid in the food 2 to be misted has decreased to the amount of liquid not suitable for the generation of the mist.

### Second Embodiment

### 2.1. Configuration Example

Next, a mist generating device used in a second embodiment of the present disclosure will be described with reference to FIG. 9. FIG. 9 is a see-through perspective diagram illustrating an example of a configuration of a mist generating device 20 used in the present embodiment.

As illustrated in FIG. 9, the mist generating device 20 includes the oscillation tank 11, a plurality of food tanks 12A, and the ultrasonic oscillator 13. Unlike the mist generating device 10 used in the first embodiment, the mist generating device 20 used in the second embodiment has the plurality of food tanks 12A for independently generate the mists of the respective plurality of foods 2. Although FIG. 9 illustrates the mist generating device 20 including two food tanks 12A, the number of food tanks 12A included in the mist generating device 20 is not limited to two, and may be three or more.

The plurality of food tanks 12A has a structure obtained by dividing a container-like structure having the area of a bottom part smaller than that of the oscillation tank 11, and are provided inside the oscillation tank 11 so that the bottom part is in contact with the liquid 15 (not illustrated) inside the oscillation tank 11. Foods 2 different from each other are stored inside the plurality of food tanks 12A, and the plurality of food tanks 12A are provided such that the mist of the food 2 is generatable independently of each other by the oscillators of the ultrasonic oscillators 13 provided below the plurality of food tanks 12A.

In FIG. 9, each of the plurality of food tanks 12A is provided with one oscillator of the ultrasonic oscillator 13, but the present embodiment is not limited to this example. For example, the mist generating device 20 may be provided with a plurality of oscillators having different frequencies of the ultrasonic waves to be generated from each other for each of the plurality of food tanks 12A so that the ultrasonic waves of multiple frequencies are generatable independently in each of the food tanks 12A.

According to the mist generating device 20 having the above configuration, the control device 100 can generate the mist of the plurality of foods 2 from the plurality of food tanks 12A while changing the mist in accordance with time. For example, the control device 100 can mix a mist of a second food with a mist of a first food later, or can continuously change the mist to be generated from the mist of the first food to the mist of the second food. Therefore, the control device 100 and the mist generating device 20 according to the present embodiment can provide the user with a more entertaining eating and drinking experience.

### 2.2. Operation Example

Subsequently, a control in the mist generating device 20 will be described with reference to FIGS. 10 and 11. FIG. 10 is a graph illustrating an example of changing an amount of mist generated from the plurality of foods 2 on a time axis. FIG. 11 is a flowchart illustrating an example of a control in the mist generating device 20.

In the following, the control in the mist generating device 20 will be described by exemplifying a control example in which the mist of the first food (a food A) and the mist of the second food (a food B) are generated with a time lag. Specifically, as illustrated in FIG. 10, a control example is described in which, after generating the mist of the first food (the food A) at time t0, the mist of the second food (the food B) is generated at time t1, following which the mist of the first food (the food A) is stopped at time t2 and then the mist of the second food (the food B) is stopped at time t3.

In such a case, for example, between the time t0 and the time t1, the user perceives only the flavor of the mist of the food A, and between the time t1 and the time t2, the user perceives the mixed flavor of the mists of the food A and the food B. During the time t2 to the time t3, the user can only perceives the flavor of the mist of the food B. That is, the user can sequentially enjoy the mist of different flavor over time.

As illustrated in FIG. 11, first, the mist generating device 20 acquires time control data relating to the generation of the mists of the food A and the food B from the control device 100 (S301).

Next, a mode of a system is checked (S302), and if the mode of the system is in the mist generation OFF state (Mist OFF), it is checked whether or not the mist ON request flag (reqMistON) is set (S303). If the mist ON request flag is set (S303/Yes), after the mist ON request flag is cleared (reqMistON=false, S304), the mode of the system is set to the mist generation ON state (Mist ON) (S305), and a timer for measuring a passage of time is started (S306).

A control flow then returns to step S302 and the mode of the system is checked (S302). If the mode of the system is in the mist generation ON state (Mist ON), it is checked whether or not the mist OFF request flag (reqMistOFF) is set (S313). If the mist OFF request flag is not set (S313/No), a count of the timer is checked (S324).

If the timer counts the time t0, a circuit for generating the mist of the food A is controlled to be turned on (S325), and the generation of the mist of the food A is started. The control flow then returns to step S302 to resume the control.

Thereafter, if the timer count reaches the time t1, a circuit for generating the mist of the food B is controlled to be turned on (S326), and the generation of the mist of the food B is started. The control flow then returns to step S302 to resume the control.

Further, after that, if the timer count reaches the time t2, the circuit for generating the mist of the food A is controlled to be turned off (S327), and the generation of the mist of the food A is stopped. The control flow then returns to step S302 to resume the control.

Further, after that, if the timer count reaches the time t3, the circuit for generating the mist of the food B is controlled to be turned off (S328), and the generation of the mist of the food B is stopped. Thereafter, the control flow moves to step S331, the mode of the system is set to the mist generation OFF state (Mist OFF) (S331), and the timer count is stopped (S332).

On the other hand, if the mist OFF request flag is set in step S313 (S313/Yes), the circuits for generating the mists of the food A and the food B are both controlled to be off (S314). Thereafter, after the mist OFF request flag is cleared (reqMistOFF=false, S315), the mode of the system is set to the mist generation OFF state (Mist OFF) (S331), and the timer count is stopped (S332).

According to the above control, the control device 100 can so control the mist generating device 20 that the generation of the mists from the plurality of foods 2 changes with time.

### 3. Third Embodiment

### 3.1. Configuration Example

Subsequently, a mist generating device used in a third embodiment of the present disclosure will be described with reference to FIGS. 12A and 12B. FIG. 12A is a perspective diagram illustrating an example of a configuration of a mist generating device 30 used in the present embodiment. FIG. 12B is a perspective diagram illustrating another example of the configuration of the mist generating device 30 used in the present embodiment.

As illustrated in FIG. 12A, the mist generating device 30 includes the oscillation tank 11, the food tank 12, the ultrasonic oscillator 13, and an air blower 31. The mist generating device 30 used in the third embodiment differs from the mist generating device 10 used in the first embodiment, in that the mist generating device 30 includes the air blower 31 for controlling a flow of the mist of the food 2.

The air blower 31 controls an amount and a direction of the mist of the food 2 flowing from the mist generating device 30 by generating wind. The air blower 31 may include, for example, a fan or an actuator, and may be provided above the food tank 12. According to this, the air blower 31 blows the air to diffuse the mist accumulated inside the food tank 12, thereby allowing the mist to flow to the outside of the food tank 12.

Moreover, as illustrated in FIG. 12B, the mist generating device 30 may include a plurality of air blowers 31A. The plurality of air blowers 31A may each include, for example, the fan or the like for generating the wind, and may be provided on side surfaces of the food tank 12 so as to face each other. According to this, it is possible to control the direction in which the mist of the food 2 is caused to flow by selectively operating the plurality of air blowers 31A.

According to the mist generating device 30 having the above configuration, the control device 100 can control the amount and the direction of the mist of the food 2, so that visual entertainment can be further enhanced. For example, when a new dish or the like is placed on a table, the control device 100 can cause the mist to flow in a direction of the newly placed dish, change a direction in which the mist flows in accordance with the music playing in the surroundings, or cause the mist to start to flow at the timing at which the user is seated. These environment and user sensing may be performed by a sensor system provided in the control device 100 and the mist generating device 30, or may be performed by another sensor system that cooperates with the control device 100 and the mist generating device 30. Therefore, the control device 100 and the mist generating device 30 according to the present embodiment can provide the user with a more entertaining eating and drinking experience.

In addition, the third embodiment can be combined with the second embodiment. That is, the mist generating device 30 may include the oscillation tank 11, the plurality of food tanks 12A, the ultrasonic oscillator 13, and the air blower 31. According to this, the mist generating device 30 mixes the mists of the plurality of foods 2 at any ratio by the air blower 31, and changes the mixing ratio of the mists of the plurality of foods 2 with the passage of time. In such a case, the mist generating device 30 can provide the user with a more entertaining eating and drinking experience.

### 3.2. Operation Example

Next, a control in the mist generating device 30 will be described with reference to FIG. 13. FIG. 13 is a flowchart illustrating an example of the control in the mist generating device 30.

In the following, the control in the mist generating device 30 will be described by exemplifying a control example in which the direction of the mist flow is switched every 5 seconds. Specifically, a control example is described that controls a direction in which the mist flows by switching the air blowers 31A (a fan A and a fan B) that operate for each 5 seconds by using the mist generating device 30 having two air blowers 31A (the fan A and the fan B).

As illustrated in FIG. 13, first, a mode of a system is checked (S401), and if the mode of the system is in the mist generation OFF state (Mist OFF), it is checked whether or not the mist ON request flag (reqMistON) is set (S402). If the mist ON request flag is set (S402/Yes), the ultrasonic oscillator notified from the control device 100 is selected (S403), and the mist generating circuit is controlled to be ON (S404). Thereafter, after the mist ON request flag is cleared (reqMistON=false, S405), the mode of the system is set to the mist generation ON state (Mist ON) (S406). Subsequently, the fan A, which is one of the two air blowers 31A, is turned on (S407), and a timer for measuring elapsed time starts counting (S408).

A control flow then returns to step S401 and the mode of the system is checked (S401). If the mode of the system is in the mist generation ON state (Mist ON), it is checked whether or not the mist OFF request flag (reqMistOFF) is set (S411). If the mist OFF request flag is not set (S411/No), it is checked whether the timer count is 5 seconds or more (S422).

If the count of the timer is 5 seconds or more (S422/Yes), the ON/OFF states of the fan A and the fan B as the two air blowers 31A are switched (S423). Specifically, if the fan A is on, the fan A is controlled to be off, and the fan B is controlled to be on. Further, if the fan B is on, the fan A is controlled to be on and the fan B is controlled to be off. Subsequently, the timer count is reset to 0 (S424). The control flow then returns to step S401 and the mode of the system is checked (S401).

On the other hand, if the mist OFF request flag is set in step S411 (S411/Yes), the mist generating circuit is controlled to be turned off (S412). Thereafter, after the mist OFF request flag is cleared (reqMistOFF=false, S413), the mode of the system is set to the mist generation OFF state (Mist OFF) (S414). Subsequently, the fan A and the fan B, which are the two air blowers 31A, are controlled to be turned off (S415), and the counting of the timer is stopped (S416).

According to the above control, the control device 100 can control the mist generating device 30 so that the two air blowers 31A (the fan A and the fan B) switch the direction in which the mist from the food 2 flows every 5 seconds.

### 3.3. Modification Example

Subsequently, a modification example of the present embodiment will be described with reference to FIG. 14. FIG. 14 is an explanatory diagram for describing a modification example of the present embodiment.

The control device 100 can provide the user with a more complicated eating and drinking experience by using a plurality of mist generating devices 30A and 30B.

Specifically, as illustrated in FIG. 14, a user 5A is provided with the mist generating device 30A, and a user 5B is provided with the mist generating device 30B. The mist generating device 30A and the mist generating device 30B are respectively provided with the fan A and the fan B as the air blowers 31A, the fan A can generate wind in a direction from the user 5B to the user 5A, and the fan B can generate wind in a direction from the user 5A to the user 5A.

Here, at the beginning of the mist generation, the fan A and the fan B of the mist generating device 30A and the mist generating device 30B are controlled to be off. Thereby, the user 5A can enjoy the mist 1A generated from the mist generating device 30A provided to himself/herself, and the user 5B can enjoy the mist 1B generated from the mist generating device provided to himself/herself.

Thereafter, for example, the fan B of the mist generating device 30A is controlled to be on, and the fan A of the mist generating device 30B is controlled to be on. Thereby, the user and the user 5B can enjoy a more complicated aroma and flavor in which the mist 1A generated from the mist generating device 30A and the mist 1B generated from the mist generating device are mixed. Therefore, the user 5A and the user 5B can obtain a more complicated eating and drinking experience.

### 4. Fourth Embodiment

Next, a mist generating device used in a fourth embodiment of the present disclosure will be described with reference to FIGS. 15 and 16. FIG. 15 is a perspective diagram illustrating an example of a configuration of a mist generating device 40 used in the present embodiment. FIG. 16 is a schematic longitudinal sectional diagram illustrating the mist generating device 40 illustrated in FIG. 15.

As illustrated in FIGS. 15 and 16, the mist generating device 40 includes the oscillation tank 11, a base 16, the food tank 12, and the ultrasonic oscillator 13. The oscillation tank 11 contains the liquid 15 for propagating the ultrasonic waves to the food tank 12, and the food tank 12 contains the food 2 to be misted.

The mist generating device 40 used in the fourth embodiment differs from the mist generating device 10 used in the first embodiment, in that the food tank 12 is provided detachably from the oscillation tank 11.

Specifically, the food tank 12 has a container-like structure smaller than the oscillation tank 11 and is placed on the base 16 provided at the bottom part of the oscillation tank 11. The oscillation tank 11 contains the liquid 15 up to a height where the liquid 15 comes into contact with the bottom part of the food tank 12, and the ultrasonic waves generated by the ultrasonic oscillator 13 provided at the bottom part of the oscillation tank 11 propagate to the bottom part of the food tank 12 via the liquid 15. The food tank 12 may be, for example, a commercially available plastic cup made of a thin material that easily propagates the ultrasonic waves.

According to this, because the food tank 12 is provided detachably from the oscillation tank 11, it becomes possible to replace the whole food tank 12 when replacing the food 2 that generates the mist. Further, in a case where the commercially available plastic cup is used for the food tank 12, the food tank 12 is replaceable, so that a labor of cleaning the food tank 12 when replacing the food 2 can be saved.

According to the above configuration, the mist generating device 40 used in the present embodiment can save the labor involved in replacing the food 2, so that the food 2 can be replaced more easily.

### 5. Fifth Embodiment

Subsequently, a mist generating device used in a fifth embodiment of the present disclosure will be described with reference to FIGS. 17 to 19. FIG. 17 is a schematic perspective diagram illustrating an appearance of an edible member 52 containing the food 2 that generates the mist. FIG. 18 is a longitudinal sectional diagram illustrating an internal structure of the edible member 52 illustrated in FIG. 17. FIG. 19 is a see-through perspective diagram illustrating a structure of a mist generating device 50 in which the edible member 52 is placed in the oscillation tank 11.

As illustrated in FIG. 17, in the present embodiment, the edible member 52 for confectionery, cooking, or the like is used as a part of the food tank containing the liquid food 2 that generates the mist.

Specifically, as illustrated in FIG. 18, the edible member 52 may be the confectionery (so-called cake or sweets) such as savarin in which the liquid food 2 (for example, liqueur) is enclosed in an internal cavity. The edible member 52 is configured by, for example, fruits such as cherries, sponge cake, chocolate, whipped cream, etc. An inner wall of the cavity in which the food 2 of the edible member 52 is enclosed may be coated with chocolate or the like, for example, in order to prevent the liquid food 2 from soaking into the edible member 52.

As illustrated in FIG. 19, such an edible member 52 is placed inside the oscillation tank 11 containing the liquid 15 and having the ultrasonic oscillator 13 at the bottom part. As a result, the ultrasonic waves generated by the ultrasonic oscillator 13 propagate through the liquid 15 to the food 2 inside the edible member 52, so that the mist of the food 2 is generated inside the edible member 52 and the inside of the edible member 52 is filled with the mist. Therefore, the edible member 52 can perform a highly entertaining presentation in which the mist of the food 2 is released from the inside of the edible member 52 when eaten by the user. A bottom surface of the cavity inside the edible member 52 is preferably made of a thin film of food in order to propagate the ultrasonic waves generated by the ultrasonic oscillator 13.

According to the above configuration, the mist generating device 50 used in the present embodiment generates the mist of the food 2 inside the edible member 52, thereby providing the user with a more entertaining eating and drinking experience.

However, the edible member 52 is not limited to the confectionery such as savarin described above. The edible member 52 may be, for example, fruits such as lemons or various foods such as eggs, as long as the bottom part can propagate the ultrasonic waves generated by the ultrasonic oscillator 13.

### 6. Sixth Embodiment

Next, a control device 100 according to a sixth embodiment of the present disclosure will be described with reference to FIG. 20. FIG. 20 is an explanatory diagram illustrating a control by the control device 100 according to the present embodiment.

As illustrated in FIG. 20, the control device 100 may control a projection display device 600 and a mist generating device 60 by combining the projection display device 600 and the mist generating device 60 with each other.

Specifically, the control device 100 may control the generation of the mist 1 from the mist generating device 60 on the basis of the timing of projection of an image from the projection display device 600. According to this, the control device 100 can display an image using the mist 1 as a screen by projecting the image on the mist 1 generated from the mist generating device 60. That is, the control device 100 can perform so-called projection mapping on the mist 1 from the projection display device 600.

Further, the control device 100 may control the generation of the mist 1 from the mist generating device 60 on the basis of a sensing result of a user 5, a surrounding environment, and the mist 1. For example, the control device 100 may generate the mist 1 from the mist generating device 60 at the timing when the user 5 comes in front of the mist generating device 60, may generate the mist 1 from the mist generating device 60 at the timing when music starts, or may suppress the generation of the mist 1 from the mist generating device 60 in a case where the diffusion of the mist 1 from the mist generating device 60 is weak.

Furthermore, the control device 100 may control the image to be projected from the projection display device 600 on the basis of the sensing result of the user 5, the surrounding environment, and the mist 1. For example, the control device 100 may change the image to be projected from the projection display device 600 at the timing when the user 5 attaches the mist 1 to the secondary ingredient 3, may change the image to be projected from the projection display device 600 in accordance with the music playing in the surroundings, or may change the image to be projected from the projection display device 600 on the basis of a flow of the mist 1.

According to the above configuration, the control device 100 according to the present embodiment can provide the user 5 with an eating and drinking experience in which the mist 1 and the image are combined. Therefore, the control device 100 according to the present embodiment can provide the user with a more entertaining eating and drinking experience.

### 7. Hardware Configuration Example

Furthermore, with reference to FIG. 21, a hardware configuration of the control device 100 according to each embodiment of the present disclosure will be described. FIG. 21 is a block diagram illustrating a hardware configuration example of the control device 100 according to the present embodiment.

A function of the control device 100 according to the present embodiment can be achieved by cooperation between software and hardware described below. The function of the controller 120 may be executed by a CPU 901, for example. A function of the input unit 110 may be performed by an input device 915, for example.

As illustrated in FIG. 21, the control device 100 includes the CPU (Central Processing Unit) 901, a ROM (Read Only Memory) 903, and a RAM (Random Access Memory) 905.

In addition, the control device 100 may further include a host bus 907, a bridge 909, an external bus 911, an interface 913, an input device 915, an output device 917, a storage device 919, a drive 921, a connection port 923, or a communication device 925. Further, the control device 100 may include an imaging device 933 or a sensor 935 on an as-necessary basis. The control device 100 may have a processing circuit such as a DSP (Digital Signal Processor) or an ASIC (Application Specific Integrated Circuit) instead of the CPU 901 or together with the CPU 901.

The CPU 901 functions as an arithmetic processing device or a control device, and controls an operation within the control device 100 in accordance with various programs recorded in the ROM 903, the RAM 905, the storage device 919, or a removable recording medium 927. The ROM 903 stores the program to be used by the CPU 901, calculation parameters, etc. The RAM 905 temporarily stores the program to be used in the execution of the CPU 901, parameters to be used in the execution, etc.

The CPU 901, the ROM 903, and the RAM 905 are interconnected by a host bus 907, which is an internal bus such as a CPU bus. Further, the host bus 907 is coupled via a bridge 909 to the external bus 911 such as a PCI (Peripheral Component Interconnect/Interface) bus.

The input device 915 is, for example, a device such as a mouse, a keyboard, a touch panel, a button, a switch, or a lever that receives an input from the user. Note that the input device 915 may be a microphone or the like that detects the user's voice. The input device 915 may be, for example, a remote control device using infrared rays or other radio waves, or may be an external connection device 929 corresponding to an operation of the control device 100.

The input device 915 further includes an input control circuit that outputs, to the CPU 901, an input signal generated on the basis of information inputted by the user. By operating the input device 915, the user can input various pieces of data to the control device 100 or instruct processing operations.

The output device 917 is a device capable of visually or audibly presenting, to the user, information acquired or generated by the control device 100. The output device 917 is, for example, an LCD (Liquid Crystal Display), a PDP (Plasma Display Panel), an OLED (Organic Light Emitting Diode) display, a hologram, a display device such as a projector, a sound output device such as a speaker or headphones, or a printing device such as a printer. The output device 917 can output information obtained by the processing of the control device 100 as a text or a picture such as an image, or voice or sound such as audio.

The storage device 919 is a data storage device configured as an example of a storage unit of the control device 100. The storage device 919 may be configured by, for example, a magnetic storage device such as an HDD (Hard Disk Drive), a semiconductor storage device, an optical storage device, or a magneto-optical storage device. The storage device 919 can store the program to be executed by the CPU 901, various pieces of data, or various pieces of data acquired from the outside.

The drive 921 is a device for reading or writing a removable recording medium 927 such as a magnetic disk, an optical disk, a magneto-optical disk, or a semiconductor memory, and is built in or externally attached to the control device 100. For example, the drive 921 can read out information recorded on the attached removable recording medium 927 and output the information to the RAM 905. The drive 921 can also write records to the attached removable recording medium 927.

The connection port 923 is a port for directly coupling the external connection device 929 to the control device 100. The connection port 923 may be, for example, a USB (Universal Serial Bus) port, an IEEE1394 port, or a SCSI (Small Computer System Interface) port. In addition, the connection port 923 may be an RS-232C port, an optical audio terminal, an HDMI (registered trademark) (High-Definition Multimedia Interface) port, or the like. The connection port 923 is coupled to the external connection device 929 so that various pieces of data can be transmitted and received between the control device 100 and the external connection device 929.

The communication device 925 is, for example, a communication interface configured by a communication device for connecting to the communication network 931. The communication device 925 may be, for example, a communication card for wired or wireless LAN (Local Area Network), Bluetooth (registered trademark), or WUSB (Wireless USB). In addition, the communication device 925 may be a router for optical communication, a router for ADSL (Asymmetric Digital Subscriber Line), or a modem for various types of communication.

The communication device 925 can, for example, transmit and receive a signal to and from the Internet or other communication devices using a predetermined protocol such as TCP/IP. The communication network 931 coupled to the communication device 925 is a wired or wireless network, such as an Internet communication network, a home LAN, an infrared communication network, a radio wave communication network, or a satellite communication network.

It is also possible to create a program for causing the hardware such as the CPU 901, the ROM 903, or the RAM 905 incorporated in a computer to exhibit a function equivalent to that of the control device 100 described above. It is also possible to provide a computer-readable recording medium recording the program.

A technique according to the present disclosure has been described above with reference to the first embodiment to the sixth embodiment. However, the technique according to the present disclosure is not limited to the above-described embodiments and the like, and various modifications can be made.

Further, not all of the configurations and operations described in the respective embodiments are essential to the configuration and the operation of the present disclosure. For example, among the elements in the respective embodiments, elements not recited in an independent claim based on the most generic concept of the present disclosure are to be understood as optional components.

The terms used throughout this specification and the appended claims should be construed as “non-limiting” terms. For example, the terms “including” or “included” should be construed as “not being limited to an embodiment in which it is described as including”. The term “has” should be construed as “not being limited to an embodiment in which it is described as having”.

The terms used in this specification are used merely for convenience of description and include terms that are not used for the purpose of limiting a configuration and an operation. For example, terms such as “right,” “left,” “up,” and “down” merely indicate a direction in the drawing being referenced. In addition, the terms “inner” and “outer” merely indicate directions toward the center of an element of interest and away from the center of the element of interest, respectively. This applies similarly to terms similar to these terms and terms having the similar meanings.

The technique according to the present disclosure may be configured as follows. According to the technique of the present disclosure including the following configuration, it is possible to mix mists generated from a plurality of foods or to generate the mist of the plurality of foods in conjunction with the production such as music or image by controlling the generation of the mist from the plurality of foods. Accordingly, the control device, the control method, and the program according to an embodiment of the present disclosure makes it possible to provide a user with a more entertaining eating and drinking experience. The effects of the technique according to the present disclosure are not necessarily limited to the effects described herein, and may be any of the effects described in the present disclosure.
(1)
   A control device including
   a controller that controls each generation of a mist from a plurality of foods.
(2)
   The control device according to (1), in which the food includes a liquid.
(3)
   The control device according to (2), in which the mist includes fine droplets of the food.
(4)
   The control device according to any one of (1) to (3), further including a mist generator, in which
   the mist generator includes:
   an oscillation tank having an ultrasonic oscillator at a bottom part and having a container-like structure containing a liquid inside; and
   a food tank in contact with the liquid at a bottom part and having a container-like structure containing the food.
(5)
   The control device according to (4), in which the ultrasonic oscillator generates the mist from the food by propagating a vibration to the food via the liquid and the food tank.
(6)
   The control device according to (4) or (5), in which the food tank includes a plurality of the food tanks containing the respective foods that are different from each other, in which
   the controller controls each of the generations of the mists of the mutually different foods from the plurality of food tanks.
(7)
   The control device according to any one of (4) to (6), in which the controller controls an oscillation frequency of the ultrasonic oscillator on the basis of a type of the food.
(8)
   The control device according to any one of (4) to (7), in which the controller controls an oscillation frequency of the ultrasonic oscillator on the basis of a type of a secondary ingredient to which the mist of the food is to be attached or which is to be immersed in the food.
(9)
   The control device according to any one of (4) to (8), in which the controller controls an oscillation timing of the ultrasonic oscillator on the basis of a type of the food.
(10)
   The control device according to any one of (4) to (9), in which the food tank is provided detachably from the mist generator.
(11)
   The control device according to any one of (1) to (10), in which the controller further controls a flow of the mist.
(12)
   The control device according to (11), in which the controller controls each of the flows of the mists from a plurality of the foods.
(13)
   The control device according to any one of (1) to (12), in which the controller controls the generation of the mist from the food on the basis of a sensing result of at least one or more of the mist, a user, or an environment.
(14)
   The control device according to any one of (1) to (13), in which the controller controls the generation of the mist from the food on the basis of at least one or more of a state or an amount of the food.
(15)
   The control device according to any one of (1) to (14), in which the controller controls the generation of the mist from the food on the basis of a projection timing of an image to be projected onto the mist.
   (16)
   The control device according to any one of (1) to (15), in which the controller controls the generation of the mist from the food on the basis of an output timing of music to be outputted to a user.
(17)
   A control method including
   controlling, with an arithmetic processing unit, each generation of a mist from a plurality of foods.
(18)
   A program that causes a computer to function as
   a controller that controls each generation of a mist from a plurality of foods.

The present application claims the benefit of Japanese Priority Patent Application JP2020-186820 filed with the Japan Patent Office on Nov. 9, 2020, the entire contents of which are incorporated herein by reference.

It should be understood by those skilled in the art that various modifications, combinations, sub-combinations and alterations may occur depending on design requirements and other factors insofar as they are within the scope of the appended claims or the equivalents thereof.

1. A control device comprising
a controller that controls each generation of a mist from a plurality of foods.

2. The control device according to claim 1, wherein the food comprises a liquid.

3. The control device according to claim 2, wherein the mist comprises fine droplets of the food.

4. The control device according to claim 1, further comprising a mist generator, wherein
the mist generator includes:
an oscillation tank having an ultrasonic oscillator at a bottom part and having a container-like structure containing a liquid inside; and
a food tank in contact with the liquid at a bottom part and having a container-like structure containing the food.

5. The control device according to claim 4, wherein the ultrasonic oscillator generates the mist from the food by propagating a vibration to the food via the liquid and the food tank.

6. The control device according to claim 4, wherein the food tank comprises a plurality of the food tanks containing the respective foods that are different from each other, wherein
the controller controls each of the generations of the mists of the mutually different foods from the plurality of food tanks.

7. The control device according to claim 4, wherein the controller controls an oscillation frequency of the ultrasonic oscillator on a basis of a type of the food.

8. The control device according to claim 4, wherein the controller controls an oscillation frequency of the ultrasonic oscillator on a basis of a type of a secondary ingredient to which the mist of the food is to be attached or which is to be immersed in the food.

9. The control device according to claim 4, wherein the controller controls an oscillation timing of the ultrasonic oscillator on a basis of a type of the food.

10. The control device according to claim 4, wherein the food tank is provided detachably from the mist generator.

11. The control device according to claim 1, wherein the controller further controls a flow of the mist.

12. The control device according to claim 11, wherein the controller controls each of the flows of the mists from a plurality of the foods.

13. The control device according to claim 1, wherein the controller controls the generation of the mist from the food on a basis of a sensing result of at least one or more of the mist, a user, or an environment.

14. The control device according to claim 1, wherein the controller controls the generation of the mist from the food on a basis of at least one or more of a state or an amount of the food.

15. The control device according to claim 1, wherein the controller controls the generation of the mist from the food on a basis of a projection timing of an image to be projected onto the mist.

16. The control device according to claim 1, wherein the controller controls the generation of the mist from the food on a basis of an output timing of music to be outputted to a user.

17. A control method comprising
controlling, with an arithmetic processing unit, each generation of a mist from a plurality of foods.

18. A program that causes a computer to function as
a controller that controls each generation of a mist from a plurality of foods.
